(19) United States
(12) Patent Application Publication
(10) Pub. No.: US 2019/0000679 A1
(43) Pub. Date: Jan. 3, 2019

(54) ADJUSTABLE MASTOID DRESSING AND METHOD

(71) Applicants: University of Maryland, Baltimore, Baltimore, MD (US); University of Maryland Medical Center, LLC, Baltimore, MD (US)

(72) Inventors: Jeffrey S. Wolf, Owings Mills, MD (US); Richard Vincent, Old Saybrook, CT (US)

(21) Appl. No.: 16/023,115

(22) Filed: Jun. 29, 2018

Related U.S. Application Data
(60) Provisional application No. 62/527,287, filed on Jun. 30, 2017.

(51) Int. Cl. A61F 13/12; A61F 5/01; A61F 5/37

(52) U.S. Cl. CPC A61F 13/12; A61F 5/3707; A61F 5/01

(57) ABSTRACT

Disclosed herein is an adjustable mastoid dressing that is adjustable to provide a secure fit of a mastoid portion to a patient's skull. The adjustable mastoid dressing includes an adjustable upper band and an adjustable lower band that together compress the mastoid region. The upper band and lower band include posterior portions that optimally engage the patient's occipital bone when tightened via manually operable tension adjusters.

### CROSS REFERENCE TO RELATED APPLICATION

This application claims the benefit of U.S. Provisional Application No. 62/527,287 titled “Adjustable Mastoid Dressing,” filed Jun. 30, 2017 by the inventors herein, which application is incorporated herein by reference in its entirety.

### FIELD OF THE INVENTION

The present invention relates generally to post-operative surgical dressings, and more particularly to a post-otologic surgical protection device employing an adjustable mastoid dressing and method of its use.

### BACKGROUND

Otologic surgery with mastoidectomy traditionally requires a pressure dressing to prevent formation of hematoma. Typical dressings include several layers of gauze to properly protect the ear while applying pressure to the post-auricular (mastoid) region that are immediately applied after surgery. These typical dressings are merely wrapped around a patient's head, such as forming a band around the forehead, the mastoid region, and generally the occipital bone, and are held on the patient's head by tension of the wrapping and/or a fastener.

These typical dressings have several drawbacks. For example, typical wrapped mastoid dressings are time consuming to apply (e.g., 3-5 minutes per wrapping), particularly in an expensive operating room setting during a sensitive period of a case (e.g., while the patient is waking up). Furthermore, mastoid dressings that are too loose can fall off immediately following the operation and/or when the patient leaves the hospital or clinic. However, applying a typical dressing too tightly can cause discomfort and lead to pressure necrosis of the patient's auricle and/or forehead. Mastoid wrappings that are too loose or too tight must be removed and reapplied, which often requires further visits to the provider.

### SUMMARY OF THE INVENTION

Disclosed herein is an adjustable mastoid dressing that is adjustable to provide a secure fit of a mastoid portion to a patient's skull, and a method for using such an adjustable mastoid dressing. The adjustable mastoid dressing includes an adjustable upper band and an adjustable lower band that together compress the patient's mastoid region. The upper band and lower band include posterior portions that optimally engage the patient's occipital bone when tightened via manually operable tension adjusters. The adjustable mastoid dressing offers shorter installation time than previously known methods for mastoid dressings, and likewise avoids the need to post-operatively replace or adjust a wound mastoid dressing because it was put on too loosely or too tightly. Separately adjustable upper and lower bands allow for appropriate pressure to be applied to the mastoid portion to prevent hematoma while avoiding excess pressure at pinpoint areas. The adjustable mastoid dressing may also avoid complications associated with traditional, wrapped mastoid dressings, including folding of the ear generating pressure necrosis, and/or excessive pressure over the forehead generating local tissue injury.

In accordance with certain aspects of an embodiment of the invention, an adjustable mastoid dressing is provided comprising: an adjustable upper band having a frontal portion, an upper band posterior portion, and side portions extending between the frontal portion and the upper band posterior portion; an adjustable lower band attached to the adjustable upper band and having a lower posterior portion; an upper tension adjuster operatively engaging the upper band posterior portion; a lower tension adjuster operatively engaging the lower band posterior portion; and a mastoid portion engaging the adjustable upper band and the adjustable lower band so as to be compressed by the upper band and the lower band against a patient's skull.

In accordance with further aspects of an embodiment of the invention, a method for mastoid dressing is provided, comprising the steps of: providing an adjustable mastoid dressing comprising: an adjustable upper band having a frontal portion, an upper band posterior portion, and side portions extending between the frontal portion and the upper band posterior portion; an adjustable lower band attached to the adjustable upper band and having a lower posterior portion; an upper tension adjuster operatively engaging the upper band posterior portion; a lower tension adjuster operatively engaging the lower band posterior portion; and a mastoid portion engaging the adjustable upper band and the adjustable lower band so as to be compressed by the upper band and the lower band against a patient's skull; positioning the upper band on a patient's head so that the frontal portion extends across the patient's forehead; using the upper tension adjuster to tighten the upper band against an upper portion of the patient's occipital bone; and using the lower tension adjuster to tighten the lower band against a lower portion of the patient's occipital bone.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1 is a schematic view of an adjustable mastoid dressing applied to a patient's head in accordance with certain aspects of an embodiment of the invention.

FIG. 2 is a side view of an adjustable mastoid dressing applied to a patient's head in accordance with further aspects of an embodiment of the invention.

FIG. 3 is a side view of the adjustable mastoid dressing applied to a patient's head of FIG. 2 with a mastoid portion removed for clarity.

FIG. 4 is a front view of the adjustable mastoid dressing of FIG. 3.

FIG. 5 is a rear view of the adjustable mastoid dressing of FIG. 3.

FIG. 6 is a top view of the adjustable mastoid dressing of FIG. 3.

FIG. 7 is a side view of the adjustable mastoid dressing of FIG. 3.

FIGS. 8a and 8b are side and bottom views, respectively, of a mastoid portion for use with the adjustable mastoid dressing of FIGS. 2-7.

### DETAILED DESCRIPTION OF THE PREFERRED EMBODIMENTS

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced item.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

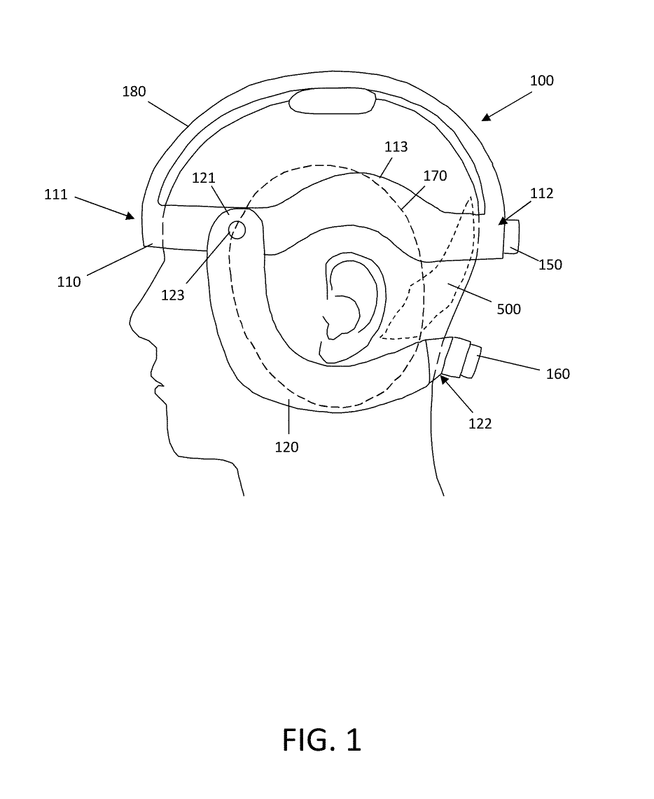

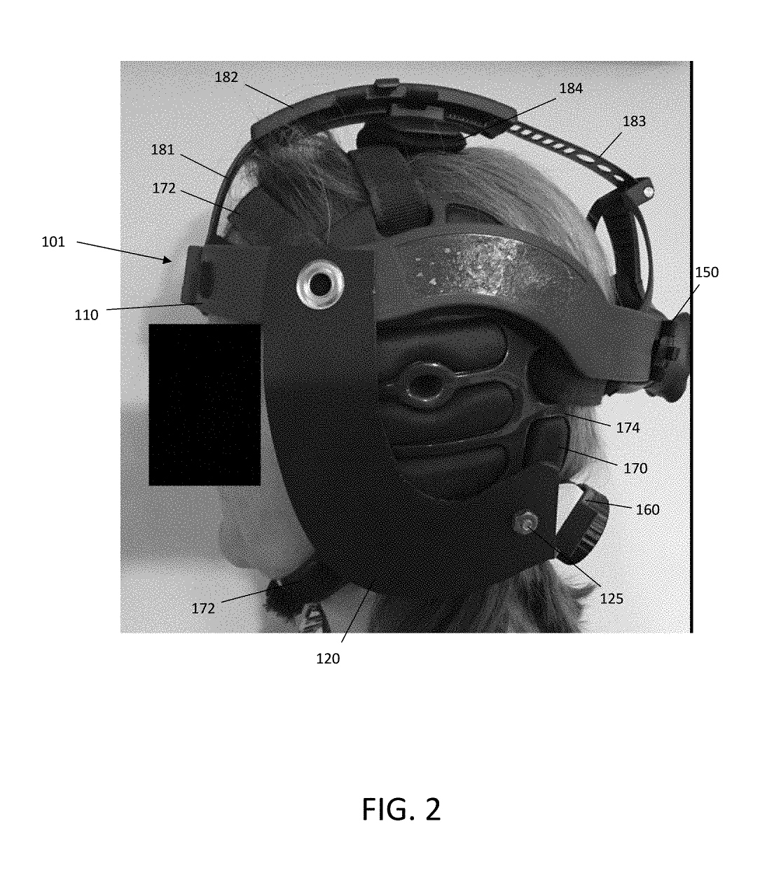

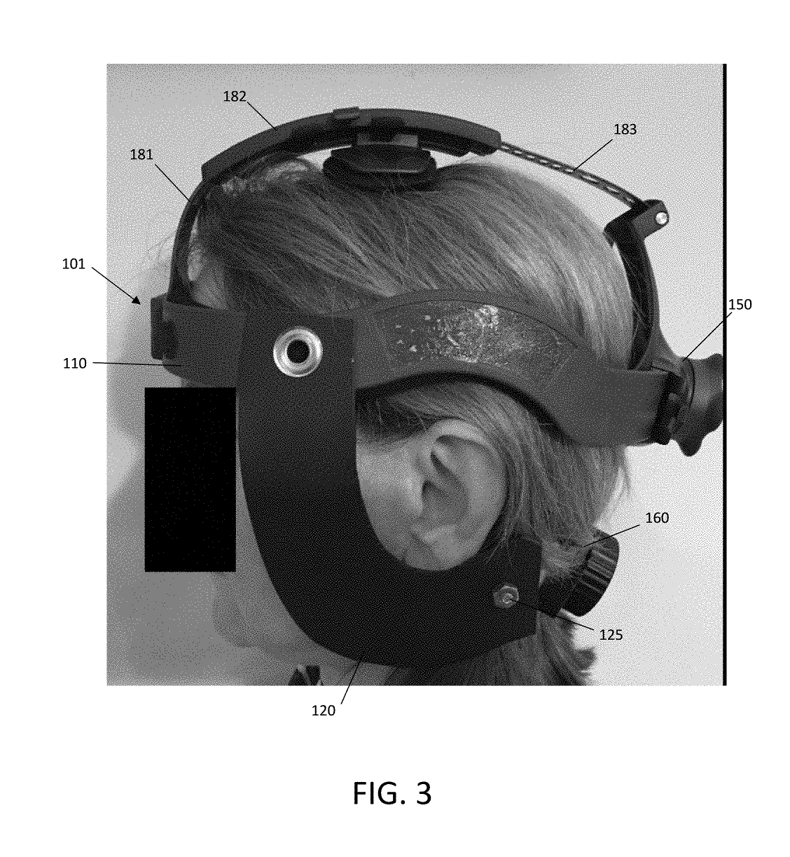

FIG. 1 is a schematic view of a frame of an adjustable mastoid dressing (shown generally at 100) in accordance with certain aspects of an embodiment of the invention. Likewise, and in accordance with a particularly preferred embodiment, FIG. 2 provides a side view of a complete adjustable mastoid dressing 101 applied to a patient's head and holding a mastoid portion 170 in place and in compression against the side of the patient's head, while FIG. 3 provides a side view of the adjustable mastoid dressing 101 of FIG. 2 with mastoid portion 170 removed for clarity.

As shown in FIGS. 1-3, in general, frame 100 of the adjustable mastoid dressing includes an upper adjustable band 110 and a lower adjustable band 120. Each of upper adjustable band 110 and lower adjustable band 120 are adjustable via tension adjusters 150 and 160, respectively, which may be adjusted in length to increase or decrease the compressive force applied to mastoid portion 170. The adjustable mastoid dressing 101 may be preassembled to reduce the time required to apply the mastoid dressing 101 to the patient. Further, the adjustable mastoid dressing 101 is configured to be simply and independently adjusted, such that it can be adjusted in the immediate post-operative period by the physician or by the patient after they leave the hospital.

With reference particularly to FIG. 1, upper adjustable band 110 includes a frontal portion 111, a posterior portion 112, and a side portion 113 extending between frontal portion 111 and posterior portion 112, with tension adjuster 150 engaging upper adjustable band 110 at the posterior portion 112. Likewise, lower adjustable band 120 includes an attaching portion 121 that connects lower adjustable band 120 to upper adjustable band 110, and a lower band posterior portion 122. Tension adjuster 160 engages lower adjustable band 120 at the lower band posterior portion 122. Attaching portion 121 of lower adjustable band 120 preferably pivotably mounts lower adjustable band 120 to upper band 110 at pivot connection 123, such that lower adjustable band 120 may pivot with respect to upper band 110 so as to adapt adjustable mastoid dressing 101 to varying patient physiologies.

As shown in FIGS. 1-3, frame 100 of adjustable mastoid dressing 101, and more particularly each of upper band 110 and lower band 120, wrap around mastoid dressing 170 on one side of the patient's skull, around the occipital bone 500 of the patient's skull, and around to the other side of the patient's skull. Upper band tension adjuster 150 is independently adjustable to modify the tension of upper band 110 on the patient's skull, while lower band tension adjuster 160 is independently adjustable to modify the tension of lower band 120 on the patient's skull. Upper band 110 is configured to be oriented substantially horizontally or in a slightly superior orientation with respect to the patient's occipital bone 500 (FIG. 1), such that upper band 110 tightens on the patient's skull preferably in a partially inferior direction and an anterior direction. Each of upper band 110 and lower band 120 may be formed of plastic, although other materials (e.g., metals, ceramics, composites, etc.) may likewise be used to fit the preference or purpose of the operator without departing from the spirit and scope of the invention. Likewise, mastoid portion 170 (FIG. 2) may optionally be formed, by way of non-limiting example, of a transparent material in order to allow the patient's ear to remain visible under the mastoid portion 170.

With continued reference to FIGS. 1-3, each of upper band 110 and lower band 120 is independently adjustable via upper tension adjuster 150 and lower tension adjuster 160, respectively, to adjust the amount and the direction of tension of the adjustable mastoid dressing 101 on the patient's head. More particularly, upper band 110 may be adjusted via upper tension adjuster 150 to pull adjustable mastoid dressing 101 partially upward/superiorily, while lower band 120 may be adjusted via lower tension adjuster 160 to pull adjustable mastoid dressing 101 partially downward/inferiorily, with both the upper band posterior portion 112 and lower band posterior portion 122 tightening against the upper and lower portions of the patient's occipital bone 500, respectively.

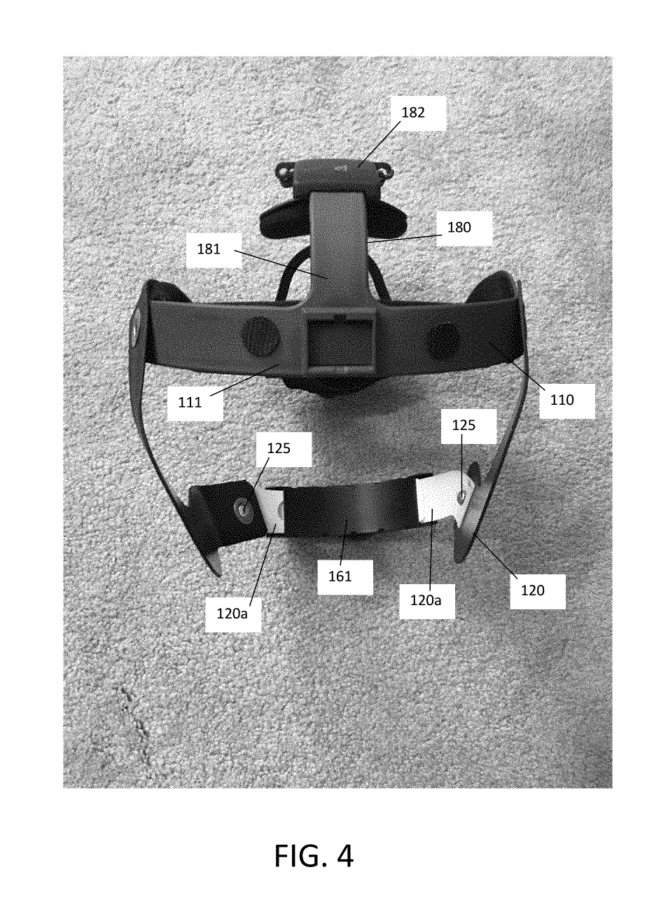

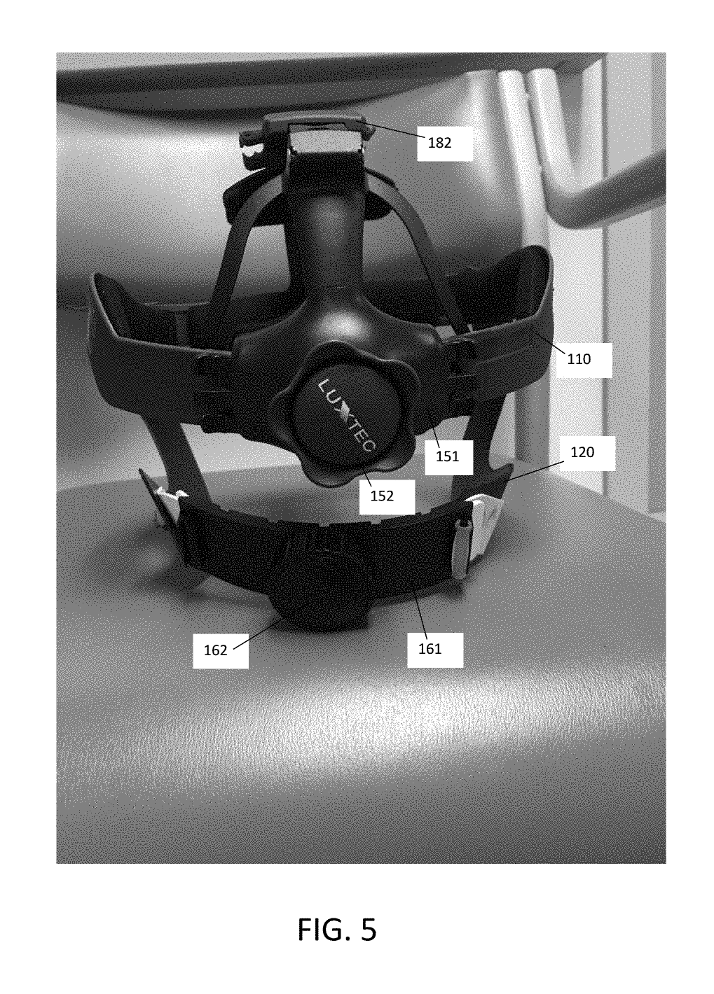

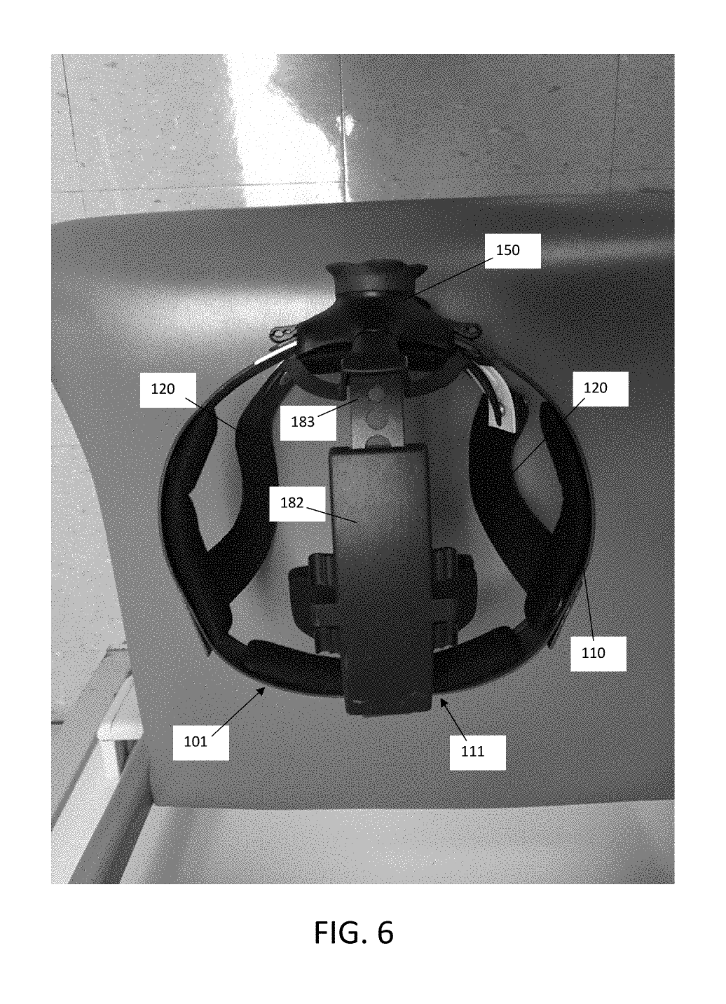

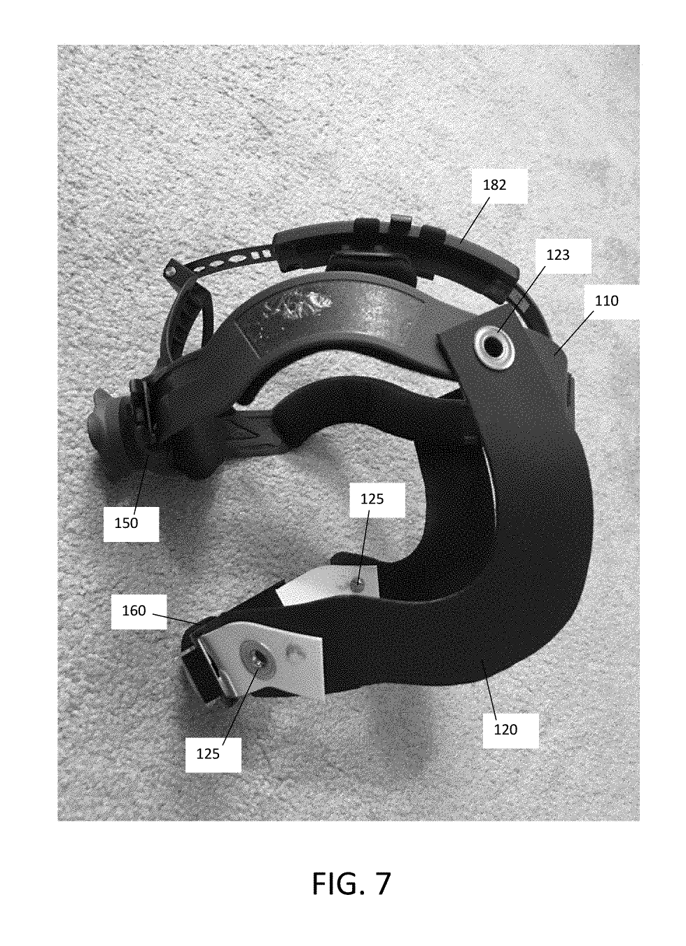

Tension adjusters 150 and 160 are configured to quickly and precisely adjust the tension (e.g., hoop stress) in the adjustable mastoid dressing 101. The adjustable mastoid dressing 101 may employ various types of tension adjusters, such as ratchets, clamps, snaps, buttons, belt-type, hook-and-loop, double adjuster, and the like, the configurations of which are well known to those of ordinary skill in the art. In the exemplary embodiment shown in FIGS. 1-7, upper tension adjuster 150 may comprise a rotating clamp including a housing 151 that receives ends of upper band 110, and a manually adjustable wheel 152 that may be rotated to either increase or decrease the length (i.e., diameter) of upper band 110. Likewise, lower tension adjuster 160 may comprise a rotating clamp including a housing 161 that receives ends of lower adjustable band 120 (see FIG. 4), and a manually adjustable wheel 162 that may be rotated to either increase or decrease the length of lower band 120. Optionally, lower adjustable band 120 may include end segments 120a that may be pivotably mounted at one end 125 to a lower portion of adjustable band 120, and may engage housing 161 at the opposite end, thus allowing fine adjustment and positioning of lower tension adjuster 160 with respect to the lower end of the patient's occipital bone 500. Those skilled in the art will recognize that alternative tension adjusting mechanisms may be used, so long as they allow independent tensioning about the upper and lower regions of the patient's occipital bone while holding mastoid portion 170 in its intended position with respect to the patient's head, without departing from the spirit and scope of the invention.

As shown in FIGS. 2-8, frame 100 of adjustable mastoid dressing 101 may also include a preferably adjustable top strap 180 that is positioned to extend over the top of a patient's head, connecting frontal portion 111 of upper adjustable band 110 to posterior portion 112 of upper adjustable band 110. Top strap 180 aids in stabilizing adjustable mastoid dressing 101 to prevent adjustable mastoid dressing 101 from falling off of the patient's head, or from moving away from the desired position. With particular reference to FIG. 4, top strap 180 includes a front portion 181 that is affixed at a first end to frontal portion 111 of upper adjustable band 110, and that joins at the opposite end to top strap housing 182. A rear top adjustment strap 183 extends rearward from top strap housing 182 and engages a top end of housing 151 of upper tension adjuster 150. Top strap housing 182 may include, by way of non-limiting example, a ratchet mechanism that engages rear top adjustment strap 183 to allow selective lengthening and shortening of top strap 180 to best fit the patient's head. Likewise, a tension adjuster similar in form to tension adjusters 150 and 160 described above may likewise be used. Further, a cushioning pad 184 may be mounted to the underside of top strap housing 182 for added comfort for the patient, which pad 184 may position top strap 182 above and off of the top of the patient's head.

In certain configurations, either or both of tension adjusters 150 and 160 may alternatively be removable, such that a physician may remove the tension adjuster 150 or 160 after tightening the adjustable mastoid dressing 101 and replace the tension adjuster 150 or 160 if tension of the adjustable mastoid dressing 101 must be adjusted (e.g., increased tension or decreased tension).

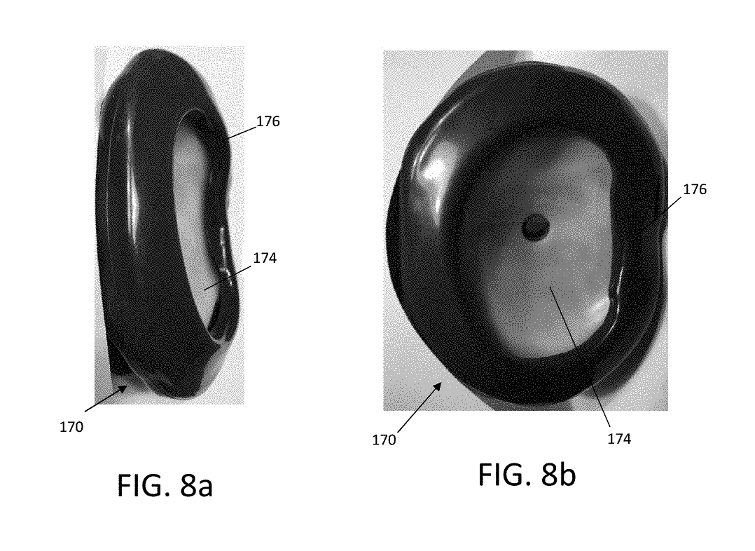

With particular reference to FIG. 2, mastoid portion 170 is configured to cover and protect a mastoid bone of the patient's skull. Further, the mastoid portion 170 is configured to substantially uniformly transform the tension of the upper adjustable band 110 and the lower adjustable band 120 into pressure exerted on the patient's skull (e.g., normal force exerted on an area of the patient's skull). As shown in FIG. 2, mastoid portion 170 may be first positioned against the user's head to cover, for example, an otologic surgical site, and may then be compressed by upper adjustable band 110 and lower adjustable band 120 of frame 100 to form a protective shell around that surgical site. Optionally, straps 172 may be provided to aid in initially positioning mastoid portion 170 against the patient's head. With continuing reference to FIG. 2 and the close-up views of FIGS. 8a and 8b, mastoid portion 170 includes an outer shell 174 forming a generally hemispherical shell-like shape, and a contact surface that presses on the patient's skull. The underside of mastoid portion 170 is a concave internal surface that covers the patient's mastoid and ear without pressing or touching the ear. The contact surface of mastoid portion 170 thus exerts a pressure on the patient's skull that is generally proportional to the adjusted tension in upper adjustable band 110 and lower adjustable band 120. In particular, the contact surface of mastoid portion 170 includes portions of contact surfaces that may exert different forces on the patient's skull. For instance, a bottom edge region of mastoid portion 170 and an upper edge region of mastoid portion 170 may exert different forces on the patient's skull, which depend, for example, on the tension in each of upper adjustable band 110 and lower adjustable band 120, the shape of the patient's head, and the shape of the mastoid portion 170. Preferably, mastoid portion 170 includes padding that is configured to protect and secure the patient's ear without discomforting the patient or damaging the patient's ear during long periods of use. For example, padding in the form of fluffs, such as unfolded 4″ by 4″ pads, cotton balls, foam, gel, and the like may be placed into the concave underside of mastoid portion 170 and against the patient's head.

FIGS. 8a and 8b show a side and bottom view, respectively, of an exemplary mastoid portion 170. Frame 100 of adjustable mastoid dressing 101 is configured to receive and hold either one such mastoid portion 170 on only one side of the patient's skull, or alternatively two such mastoid portions 170 (one on either side of the patient's skull), through simple adjustment of adjustable upper band 110 and adjustable lower band 120.

Optionally, mastoid portion 170 may be affixed to frame 100 of adjustable mastoid dressing 101, such as by way of fasteners (e.g., screws), loops, snaps, or the like, without departing from the spirit and scope of the invention.

Thus, the present invention provides an adjustable mastoid dressing that is configured to be preassembled with multiple tension adjusters. The adjustable mastoid dressing includes a frontal portion, at least one mastoid portion, a plurality of tension adjusters, and a posterior portion. The adjustable mastoid dressing is preassembled to reduce the time required to apply the mastoid dressing to the patient. The adjustable mastoid dressing with multiple tension adjusters is configured to be simply adjusted, such that it can be adjusted in the immediate post-operative period by the physician or by the patient after they leave the hospital.

While preferred embodiments of the present invention have been shown and described herein, it will be obvious to those skilled in the art that such embodiments are provided by way of example only. Numerous variations, changes, and substitutions will now occur to those skilled in the art without departing from the invention. For example, while lower adjustable band 120 is shown having a configuration that extends downward from pivot connection 123 (located at approximately a patient temple aligning position on upper band 110) so as to extend under the patient's ear to lower band posterior portion 122, it may alternatively extend in a more straight, linear fashion extending in a direction over the patient's ear while still aligning the lower band posterior portion 122 over the bottom of the patient's occipital bone. Likewise, while upper band 110 and lower band 120 are each shown with a single tension adjuster 150 and 160, respectively, either or both of upper band 110 and lower band 120 may include multiple tension adjusters to provide additional control over the application of compressive forces to the mastoid portion 170. Likewise, in addition to tension adjuster 160 on lower band 120 (or even possibly in place thereof), lower band 120 may be attachable to upper band 110 at various positions along the length of lower band 120. For instance, the ends of lower band 120 that attach to upper band 110 may be equipped with multiple first halves of snap (such as on a baseball cap) or button (such as on an outerwear jacket) attachment devices, with the mating second half of such attachment devices affixed to upper band 110, thus allowing the attachment point between upper band 110 and lower band 120, and thus the effective length of lower band 120, to vary.

It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An adjustable mastoid dressing comprising:
an adjustable upper band having a frontal portion, an upper band posterior portion, and side portions extending between the frontal portion and the upper band posterior portion;
an adjustable lower band attached to said adjustable upper band and having a lower posterior portion;
an upper tension adjuster operatively engaging said upper band posterior portion;
a lower tension adjuster operatively engaging said lower band posterior portion; and
a mastoid portion engaging said adjustable upper band and said adjustable lower band so as to be compressed by said upper band and said lower band against a patient's skull.

2. The adjustable mastoid dressing of claim 1, wherein said upper tension adjuster is configured to tension said upper band about the patient's skull with said frontal portion aligned with the patient's forehead and the upper band posterior portion aligned with an upper portion of the patient's occipital bone.

3. The adjustable mastoid dressing of claim 2, wherein said lower tension adjuster is configured to tension said lower band about a portion of the patient's skull with said lower band posterior portion aligned with a lower portion of the patient's occipital bone.

4. The adjustable mastoid dressing of claim 3, wherein said lower tension adjuster further comprises a housing receiving portions of said lower band and a manually adjustable knob engaging said portions of said lower band so as to selectively shorten and lengthen said lower band.

5. The adjustable mastoid dressing of claim 2, wherein said upper tension adjuster further comprises a housing receiving portions of said upper band and a manually adjustable knob engaging said portions of said upper band so as to selectively shorten and lengthen said upper band.

6. The adjustable mastoid dressing of claim 1, wherein said lower adjustable band is pivotably attached to said upper adjustable band.

7. The adjustable mastoid dressing of claim 1, further comprising a top strap extending upward from said frontal portion of said upper adjustable band and to said upper band posterior portion.

8. The adjustable mastoid dressing of claim 5, further comprising a cushion mounted to an underside of said top strap.

9. The adjustable mastoid dressing of claim 6, wherein said top strap has an adjustable length.

10. The adjustable mastoid dressing of claim 1, wherein said side portions of said upper adjustable band are upwardly curved to extend over a patient's ear.

11. A method for mastoid dressing, comprising the steps of:
providing an adjustable mastoid dressing comprising:
an adjustable upper band having a frontal portion, an upper band posterior portion, and side portions extending between the frontal portion and the upper band posterior portion;
an adjustable lower band attached to said adjustable upper band and having a lower posterior portion;
an upper tension adjuster operatively engaging said upper band posterior portion;
a lower tension adjuster operatively engaging said lower band posterior portion; and
a mastoid portion engaging said adjustable upper band and said adjustable lower band so as to be compressed by said upper band and said lower band against a patient's skull;
positioning said upper band on a patient's head so that said frontal portion extends across the patient's forehead;
using said upper tension adjuster to tighten said upper band against an upper portion of the patient's occipital bone; and
using said lower tension adjuster to tighten said lower band against a lower portion of the patient's occipital bone.

12. The method of claim 11, wherein said lower tension adjuster further comprises a housing receiving portions of said lower band and a manually adjustable knob engaging said portions of said lower band so as to selectively shorten and lengthen said lower band.

13. The method of claim 11, wherein said upper tension adjuster further comprises a housing receiving portions of said upper band and a manually adjustable knob engaging said portions of said upper band so as to selectively shorten and lengthen said upper band.

14. The method of claim 11, wherein said lower adjustable band is pivotably attached to said upper adjustable band.

15. The method of claim 11, wherein said adjustable mastoid dressing further comprises a top strap extending upward from said frontal portion of said upper adjustable band and to said upper band posterior portion.

16. The method of claim 15, wherein said adjustable mastoid dressing further comprising a cushion mounted to an underside of said top strap.

17. The method of claim 16, wherein said top strap has an adjustable length.

18. The method of claim 11, wherein said side portions of said upper adjustable band are upwardly curved to extend over a patient's ear.
